(19) United States
(12) Patent Application Publication
(10) Pub. No.: US 2025/0275903 A1
(43) Pub. Date: Sep. 4, 2025

(54) COMPOSITION COMPRISING BIOSURFACTANT AND SALICYLIC ACID

(71) Applicant: L'OREAL, Paris (FR)

(72) Inventors: Siliu TAN, Westfield, NJ (US); Hiroaki Kaga, Kawasaki (JP); Mariko Hasebe, New York, NY (US); Changlong Chen, Edison, NJ (US); Christopher Pang, New York, NY (US); Masanori Orita, Kawaski (JP)

(73) Assignee: L'OREAL, Paris (FR)

(21) Appl. No.: 18/591,178

(22) Filed: Feb. 29, 2024

(51) Int. Cl. A61K 8/368; A61K 8/60; A61Q 19/00; A61Q 19/10

(52) U.S. Cl. CPC A61K 8/368; A61K 8/602; A61Q 19/007; A61Q 19/10

(57) ABSTRACT

The present invention relates to a composition comprising (a) at least one glycolipid; (b) at least one salicylic acid or its derivatives represented by formula (I); (c) at least one cationic polymer may be a polysaccharide derived from β-linked D-glucosamine and N-acetyl-D-glucosamine; and (d) at least one thickener. The composition according to the present invention can provide an effect of treating, hydrating, and/or cleansing acne-prone skin.

### TECHNICAL FIELD

The present invention relates to a composition comprising at least one biosurfactant and at least one salicylic acid or its derivatives, preferably a cosmetic composition comprising the same.

### BACKGROUND ART

Anti-acne cleansing compositions often have the undesired effect of drying out the skin. Therefore, there is a need to develop an effective anti-acne composition which can provide sensorial and aesthetic benefits to acne prone-skin and also provide adequate hydration to the skin.

### DISCLOSURE OF INVENTION

An objective of the present invention is to provide a composition for treating, hydrating and/or cleansing acne-prone skin.

The above objective of the present invention can be achieved by a composition comprising:
(a) at least one glycolipid;
(b) at least one salicylic acid or its derivatives of formula (I):

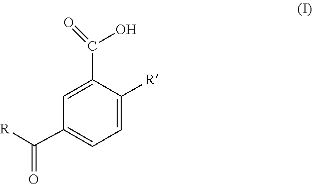

in which:
   the radical R denotes a linear, branched or cyclic, saturated aliphatic chain containing from 2 to 22 carbon atoms; an unsaturated chain containing from 2 to 22 carbon atoms containing one or more double bonds that may be conjugated; an aromatic nucleus linked to the carbonyl radical directly or via saturated or unsaturated aliphatic chains containing from 2 to 7 carbon atoms; said groups possibly being substituted with one or more substituents, which may be identical or different, chosen from halogen atoms, a trifluoromethyl group, hydroxyl groups in free form or esterified with an acid containing from 1 to 6 carbon atoms, or carboxyl groups in free form or esterified with a lower alcohol containing from 1 to 6 carbon atoms;
   R′ is a hydroxyl group or an ester group of formula:

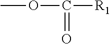

in which R₁ denotes a linear or branched, saturated or unsaturated aliphatic chain containing 1 to 18 carbon atoms;
and also salts thereof derived from an inorganic or organic base;
(c) at least one cationic polymer; and
(d) at least one thickener.

The (a) glycolipid is selected from the group consisting of rhamnolipids, sophorolipids, glucolipids, trehalolipids, cellobiose lipids and mixtures thereof.

The (b) salicylic acid derivatives are represented by formula (I) in which the radical R denotes a linear, branched or cyclic, saturated aliphatic chain containing from 2 to 22 carbon atoms.

The (b) salicylic acid derivatives are represented by formula (I) in which the R radical denotes a linear or branched alkyl or alkenyl group, preferably alkyl group, containing 2 or more, preferably 3 or more, more preferably 4 or more, even more preferably 5 or more, and in particular 6 or more carbon atoms, and/or 22 or less, preferably 18 or less, even more preferably 14 or less, preferentially 12 or less, and in particular 10 or less carbon atoms.

The (b) salicylic acid derivatives are represented by formula (I) in which R′ denotes a hydroxyl group.

The (c) cationic polymer is a polysaccharide derived from β-linked D-glucosamine and N-acetyl-D-glucosamine.

The (d) thickener is a plant-based thickener.

The amount of the (a) glycolipid(s) may be 0.1% to 10% by weight, preferably 1% to 7% by weight, more preferably 2% to 5% by weight, and in particular 3% by weight, relative to the total weight of the composition.

The amount of the (b) salicylic acid or its derivatives may be from 0.1% to 5% by weight, preferably from 1% to 3% % by weight, most preferably 2% by weight, relative to the total weight of the composition.

The weight ratio of the (a) glycolipid(s) to the (b) salicylic acid or its derivatives in the composition may be from 3:1 to 1:3, more preferably from 2:1 to 1:2, most preferably 3:2.

The composition according to the present invention may be in the forms of a solution, a cream, a lotion, an emulsion or a gel etc. Preferably, the composition of the present invention has a unique wasabi-like paste texture.

The composition may further include water in an amount of from 20% to 70% by weight, relative to the total weight of the composition.

The composition may be a cosmetic composition for treating, hydrating, and/or cleansing as acne-prone skin.

The present invention also relates to method for treating, hydrating, and/or cleansing as acne-prone skin, comprising:
applying onto the keratinous substances the composition according to the present invention.

The present invention also relates to a use of a combination of the (a) glycolipid, the (b) salicylic acid or its derivative, the (c) cationic polymer, and the (d) thickener for treating, hydrating, and/or cleansing as acne-prone skin.

### BEST MODE FOR CARRYING OUT THE INVENTION

After diligent research, the inventors have surprisingly discovered that a composition comprising (a) at least one glycolipid and (b) at least one salicylic acid or its derivatives having the specific structure of formula (I), (c) at least one cationic polymer, and (d) at least one thickener can treat, hydrate, and/or cleansing acne-prone skin, thus completing the present invention.

Thus, the composition according to the present invention is a composition comprising:
(a) at least one glycolipid;
(b) at least one salicylic acid or its derivatives of formula (I):

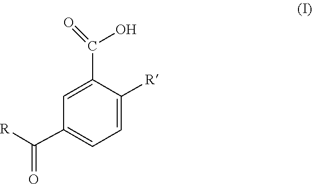

in which:
   the radical R denotes a linear, branched or cyclic, saturated aliphatic chain containing from 2 to 22 carbon atoms; an unsaturated chain containing from 2 to 22 carbon atoms containing one or more double bonds that may be conjugated; an aromatic nucleus linked to the carbonyl radical directly or via saturated or unsaturated aliphatic chains containing from 2 to 7 carbon atoms; said groups possibly being substituted with one or more substituents, which may be identical or different, chosen from halogen atoms, a trifluoromethyl group, hydroxyl groups in free form or esterified with an acid containing from 1 to 6 carbon atoms, or carboxyl groups in free form or esterified with a lower alcohol containing from 1 to 6 carbon atoms;
   R′ is a hydroxyl group or an ester group of formula:

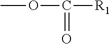

in which R₁ denotes a linear or branched, saturated or unsaturated aliphatic chain containing 1 to 18 carbon atoms;
and also salts thereof derived from an inorganic or organic base;
(c) at least one cationic polymer; and
(d) at least one thickener.

Hereinafter, the composition and the method according to the present invention will be explained in a more detailed manner.

### [Composition]

The composition may be a cosmetic composition, preferably a cosmetic composition for a keratinous substance, and more preferably a cosmetic composition for treating, hydrating, and/or cleansing acne-prove skin. The keratinous substance here means a material containing keratin as a main constituent element, and examples thereof include the facial or body skin, scalp, lips, and the like.

The form of the composition according to the present invention is not particularly limited. In general, the composition according to the present invention can be liquid, semi-liquid, or solid at room temperature (25° C.) and atmospheric pressure (10⁵ Pa). The composition may take various forms, such as a solution, an aqueous solution, a lotion, a milky lotion, a cream, a gel, a liquid gel, a paste, a serum, a suspension, a dispersion, a fluid, a milk, an emulsion (O/W or W/O form), or the like. It is preferable that the composition according to the present invention be in the form of a paste. Particularly, the composition of the present invention has a unique wasabi-like paste texture.

The composition according to the present invention may preferably be used as a cosmetic composition, in particular a rinse-off type cosmetic composition. The composition according to the present invention may be intended for application onto a keratinous substance, preferably skin, and in particular facial skin.

The composition according to the present invention comprises (a) at least one glycolipid; (b) at least one salicylic acid or its derivatives according to formula (I); (c) at least one cationic polymer, and (d) at least one thickener. The ingredients in the composition will be described in a detailed manner below.

### (Glycolipid)

The composition according to the invention comprises one or more glycolipids.

The term “glycolipid” is understood as meaning a compound formed from a lipid to which are attached one or more sugar compounds.

The one or more glycolipids may be selected from rhamnolipids, sophorolipids, glucolipids, trehalolipids, cellobiose lipids and mixtures thereof.

The one or more glycolipids are preferably selected from rhamnolipids, sophorolipids and mixtures thereof, more preferably rhamnolipids.

### Glucolipids:

The one or more glycolipids may be glucolipids, which contain a glucose moiety and can be represented by the general formula (I):

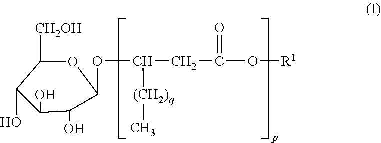

in which:
   R¹ represents a hydrogen atom or a cation,
   p denotes an integer ranging from 1 to 4, and
   q denotes an integer ranging from 4 to 10, preferably equal to 6.

The glucolipids can be produced by the bacterium Alcaligenes sp. MM 1.

The appropriate fermentation methods are reviewed by M. Schmidt in his doctoral thesis (1990), Technical University of Braunschweig, and by Schulz et al. (1991) Z. Naturforsch., 46C, 197-203. The glucolipids are recovered from the fermentation broth by solvent extraction using diethyl ether or a dichloromethane:methanol or chloroform:methanol mixture.

### Sophorolipids

The one or more glycolipids may be sophorolipids, which contain a sophorose moiety and can be represented by the general formula (II):

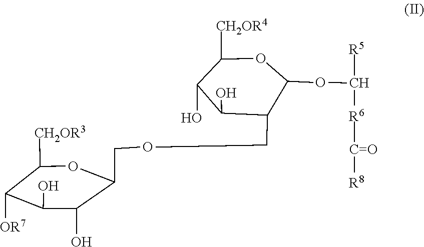

in which:
   R³ and R⁴ individually represent a hydrogen atom or an acetyl group,
   R⁵ represents a saturated or unsaturated, hydroxylated or non-hydroxylated hydrocarbon group having from 1 to 9 carbon atoms, preferably methyl,
   R⁶ represents a saturated or unsaturated, hydroxylated or non-hydroxylated hydrocarbon group having from 1 to 19 carbon atoms,
with the proviso that the total number of carbon atoms in the groups R⁵ and R⁶ does not exceed 20 and is preferably from 14 to 18.

Sophorolipids may be incorporated into the composition according to the invention either in the form of the open-chain free acid, where R⁷ represents a hydrogen atom and R⁸ represents a hydroxy group OH, or in its lactone form, where a lactone ring is formed between R⁷ and R⁸, as indicated by formula (III):

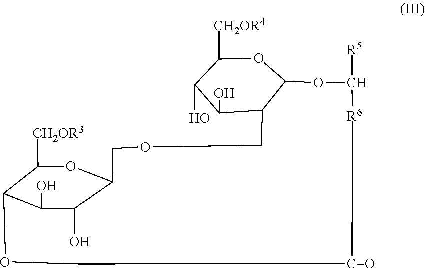

in which:
   R³, R⁴, R⁵ and R⁶ are as defined above,
with the proviso that at least one of R³ and R⁴ represents an acetyl group.

The sophorolipids can be produced by yeast cells, for example Torulopsis apicola and Torulopsis bombicola cells. The fermentation process generally uses sugars and alkanes as substrates.

Appropriate fermentation methods are reviewed in A. P. Tulloch, J. F. T. Spencer and P. A. J. Gorin, Can. J. Chem. (1962), 40, 1326, and U. Gobbert, S. Lang and F. Wagner, Biotechnology Letters (1984), 6 (4), 225. The resulting product is a mixture of various open-chain sophorolipids and of sophorolipid lactones that may be used in the form of mixtures, or the required form may be isolated.

It is possible to use as sophorolipids for example that sold under the Sopholiance S name by Givaudan and that sold under the BioToLife name by BASF.

### Trehalolipids

The one or more glycolipids may be trehalolipids, which contain a trehalose fragment and can be represented by the general formula (IV):

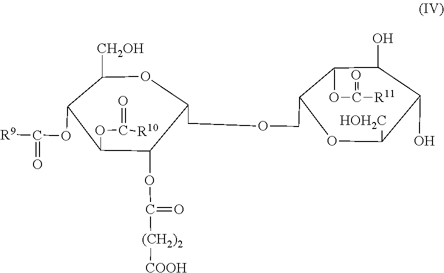

in which:
   R⁹, R¹⁰ and R¹¹ individually represent a saturated or unsaturated, hydroxylated or non-hydroxylated hydrocarbon radical having from 5 to 13 carbon atoms.

The trehalolipids can be produced by bacterial fermentation using the marine bacterium Arthrobacter sp. Ek 1 or the freshwater bacterium Rhodococcus erythropolis. Appropriate fermentation methods are provided by Ishigami et al. (1987), J. Jpn. Oil Chem. Soc., 36, 847-851, Schultz et al. (1991), Z. Naturforsch., 46C, 197-203, and Passeri et al. (1991), Z. Naturforsch., 46C, 204-209.

### Cellobiose Lipids

The one or more glycolipids may be cellobiose lipids, which contain a cellobiose fragment and can be represented by the general formula (V):

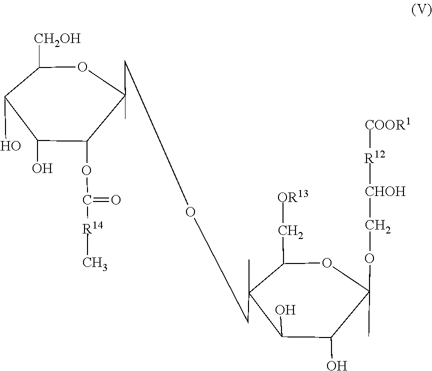

in which:
   R¹ represents a hydrogen atom or a cation,
   R¹² represents a saturated or unsaturated, hydroxylated or non-hydroxylated hydrocarbon radical having from 9 to 15 carbon atoms, preferably 13 carbon atoms,
   R¹³ represents a hydrogen atom or a acetyl group;
   R¹⁴ represents a saturated or unsaturated, hydroxylated or non-hydroxylated hydrocarbon radical having from 4 to 16 carbon atoms.

The cellobiose lipids can be produced by cells of fungi of the genus Ustilago. Appropriate fermentation processes are provided by Frantz, Lang and Wagner (1986), Biotech. Letts., 8, 757-762.

### Rhamnolipids:

The one or more glycolipids may be rhamnolipids.

The composition according to the invention preferably comprises one or more rhamnolipids.

Rhamnolipids are glycolipids produced by various bacterial species. They consist of one rhamnose fragment (mono-rhamnolipid) or of two rhamnose fragments (di-rhamnolipid) linked by a glycosidic bond to one, two or three chains of β-hydroxylated fatty acids linked to one another by an ester bond.

More specifically, these mono-rhamnolipids and di-rhamnolipids correspond to the following formula (VI):

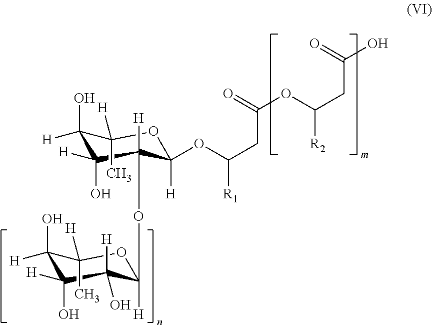

in which:
   m denotes an integer equal to 2, 1 or 0,
   n denotes an integer equal to 1 or 0, and
   R₁ and R₂, each independently represent identical or different hydrocarbon radicals having from 2 to 24 carbon atoms, preferably from 5 to 13 carbon atoms, that are branched or unbranched, substituted or unsubstituted, in particular hydroxy-substituted, and saturated or unsaturated, preferably a singly, doubly or triply unsaturated alkyl radical.

Thus, when n is equal to 0, the formula (VI) protects mono-rhamnolipids and, when n is equal to 1, it protects di-rhamnolipids.

The composition according to the invention preferably comprises at least one di-rhamnolipid.

The composition according to the invention preferably comprises at least one di-rhamnolipid of formula (VI) in which:
m denotes an integer equal to 2, 1 or 0;
n denotes an integer equal to 1; and
R₁ and R₂, each independently represent identical or different hydrocarbon radicals having from 2 to 24 carbon atoms, preferably from 5 to 13 carbon atoms, that are branched or unbranched, substituted or unsubstituted, in particular hydroxy-substituted, and saturated or unsaturated, preferably a singly, doubly or triply unsaturated alkyl radical, and also the salts thereof, solvates thereof and optical isomers thereof.

The glycosidic bond between the two rhamnose fragments may be in the alpha or beta configuration and is preferably in the alpha configuration.

In the context of the invention,
the salts of the di-rhamnolipids of formula (VI) are more particularly the carboxylate salts thereof with an organic or inorganic cation and especially with a cation selected from sodium, potassium, calcium and ammonium.
the solvated forms of the di-rhamnolipids of formula (VI) are more particularly those solvated with one or more molecules of water or of organic solvents, for example a hydrate or a solvate of a linear or branched alcohol, such as ethanol or isopropanol, the optically active carbon atoms of the fatty acids preferably being in the form of the R enantiomers, and
the term “alkyl” radical denotes a saturated, linear or branched aliphatic group; for example, a C₁-C₂₀ alkyl group having a linear or branched hydrocarbon chain of 1 to 20 carbon atoms, more particularly a methyl, ethyl, propyl, isopropyl, butyl, isobutyl, tert-butyl, pentyl, hexyl, heptyl, octyl, nonyl, decyl, undecyl, dodecyl, tridecyl, tetradecyl, pentadecyl, hexadecyl, heptadecyl, octadecyl, nonadecyl or eicosyl.

The composition according to the invention preferably comprises at least one di-rhamnolipid of formula (VI) in which:
m denotes an integer equal to 2, 1 or 0;
n denotes an integer equal to 1; and
R₁ and R₂, which are identical or different, are selected from pentenyl, hexenyl, heptenyl, octenyl, nonenyl, decenyl, undecenyl, dodecenyl and tridecenyl radicals and radicals of formula —(CH₂)ₒCH₃, with o denoting an integer ranging from 1 to 23, in particular from 3 to 15 and more particularly from 4 to 12.

According to one embodiment of the invention, the composition according to the invention comprises at least one di-rhamnolipids of general formula (VI) in which m is equal to 1

According to one embodiment of the invention, the composition according to the invention comprises a mixture of at least two, preferably at least three, di-rhamnolipids of general formula (VI) in which m is preferably equal to 1.

According to another embodiment of the invention, the composition according to the invention comprises a mixture comprising at least one mono-rhamnolipid.

More preferably, the composition according to the invention comprises at least one dirhamnolipid of the following formula (VII):

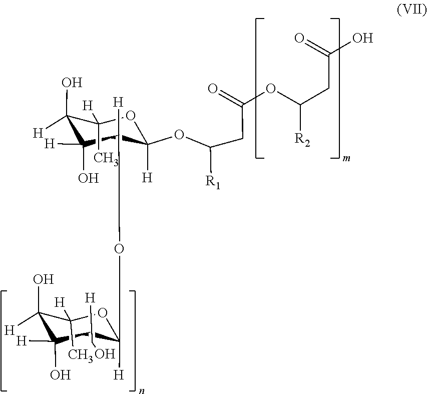

in which:
   m denotes an integer equal to 2, 1 or 0; preferably, m is equal to 1,
   n denotes an integer equal to 1,
   R₁ is a —(CH₂)ₚ—CH₃ radical, with p being an integer varying from 1 to 23, preferably from 4 to 12,
   R₂ is a —(CH₂)_{q}—CH₃ radical, with q being an integer varying from 1 to 23, preferably from 4 to 12, and also the salts thereof, solvates thereof and optical isomers thereof.

By way of illustration and without limiting the di-rhamnolipids of formula (VII) that may be suitable for the invention, mention may be made in particular of the compounds of formula di-RL-CXCY, such as are defined in Table 1 below.

The formula di-RL-CXCY is an alternative way of writing in order to represent a di-rhamnolipid (di-RL) functionalized by two radicals R₁ and R₂ respectively represented by the symbols CX and CY, the integers X and Y being respectively equal to p+4 and q+4.

TABLE 1

| Composés | Di-RL-CXCY | p | q |
|---|---|---|---|
| 1 | diRL-C8C8 | 4 | 4 |
| 2 | diRL-C8C10 | 4 | 6 |
| 3 | diRL-C10C8 | 6 | 4 |
| 4 | diRL-C10C10 | 6 | 6 |
| 5 | diRL-C10C12 | 6 | 8 |
| 6 | diRL-C12C10 | 8 | 6 |
| 7 | diRL-C12C12 | 8 | 8 |
| 8 | diRL-C12C14 | 8 | 10 |
| 9 | diRL-C14C12 | 10 | 8 |
| 10 | diRL-C14C14 | 10 | 10 |
| 11 | diRL-C14C16 | 10 | 12 |
| 12 | diRL-C16C14 | 12 | 10 |
| 13 | diRL-C16C16 | 12 | 12 |

According to a preferred embodiment, the composition according to the invention comprises at least one di-rhamnolipid of formula (VII) in which p and q are identical and equal to 6 and m is equal to 1, also referred to as di-RL-C10C10, or one of the salts, solvates and optical isomers thereof.

Preferably, the di-rhamnolipid of formula (VII) in which p and q are identical and equal to 6 and m is equal to 1 is present in the composition according to the invention in a proportion of at least 50% by weight and preferably of from 51% to 85% by weight, relative to the total weight of rhamnolipids.

According to another embodiment, the composition according to the invention comprises at least one di-rhamnolipid of formula (VII) in which m is equal to 1, p is equal to 6 and q is equal to 8.

According to another embodiment, the composition according to the invention comprises at least one di-rhamnolipid of formula (VI) in which n and m are equal to 1, R₁ represents a —(CH₂)ₒCH₃ radical, with o being an integer varying from 4 to 12, and R₂ is selected from the pentenyl, hexenyl, heptenyl, octenyl, nonenyl, decenyl, undecenyl, dodecenyl and tridecenyl radicals; preferably, R₁ represents a —(CH₂)₆CH₃ radical and R₂ a nonenyl radical.

According to another preferred embodiment, the composition according to the invention comprises a mixture of at least two, in particular at least three, di-rhamnolipids of formula (VI) or of formula (VII) selected from:
a di-rhamnolipid of formula (VII) in which p and q are identical and equal to 6 and m is equal to 1;
a di-rhamnolipid of formula (VII) in which m is equal to 1, p is equal to 6 and q is equal to 8; and
at least one di-rhamnolipid of formula (VI) in which n and m are equal to 1, R₁ represents a —(CH₂)ₒCH₃ radical, with o being an integer varying from 4 to 12, and R₂ is selected from the pentenyl, hexenyl, heptenyl, octenyl, nonenyl, decenyl, undecenyl, dodecenyl and tridecenyl radicals; preferably, R₁ represents a —(CH₂)₆CH₃ radical and R₂ a nonenyl radical.

Preferably, the composition according to the invention comprises a mixture of at least two, in particular at least three, di-rhamnolipids of formula (VI) or of formula (VII) selected from:
at least 50% by weight and preferably of from 51% to 85% by weight of a di-rhamnolipid of formula (VII) in which p and q are identical and equal to 6 and m is equal to 1, relative to the total weight of rhamnolipids.
from 0.5% to 25% by weight, preferably from 5% to 15% by weight, of a dirhamnolipid of formula (VII) in which p is equal to 6, q is equal to 8 and m is equal to 1, relative to the total weight of rhamnolipids, and
from 0.5% to 15% by weight, preferably from 3% to 12% by weight, preferably from 5% to 10% by weight, of a dirhamnolipid of formula (VI) in which n and m are equal to 1, R₁ represents a —(CH₂)₆CH₃ radical and R₂ represents a nonenyl radical, relative to the total weight of rhamnolipids.

As specified above, rhamnolipids are customarily prepared by processes known to those skilled in the art starting from bacterial producers, such as Pseudomonas.

Appropriate fermentation methods are reviewed by D. Haferburg, R. Hommel, R. Claus and H. P. Kleber in Adv. Biochem. Ing./Biotechnol. (1986), 33, 53-90, and by F. Wagner, H. Bock and A. Kretschmar in Fermentation (ed. R. M. Lafferty) (1981), 181-192, Springer Verlag, Vienna.

Use may be made, as rhamnolipid, of the one sold under the name Rheance One by Evonik (INCI name: glycolipids).

The composition according to the invention preferably comprises an amount of glycolipid ranging from 0.1% to 10% by weight, preferably from 1% to 7% by weight and even better still from 2% to 5% by weight, relative to the total weight of the cosmetic composition.

The composition according to the invention preferably comprises an amount of rhamnolipid ranging from 0.1% to 10% by weight, preferably from 1% to 7% by weight and even better still from 2% to 5% by weight, relative to the total weight of the cosmetic composition.

### (Salicylic Acid or its Derivatives)

The composition according to the present invention comprises (b) at least one salicylic acid or its derivatives according to formula (I). A single type of the salicylic acid or its derivative may be used, but two or more different types of the salicylic acid or its derivatives may be used in combination.

The salicylic derivatives in accordance with the present invention correspond to formula (I):

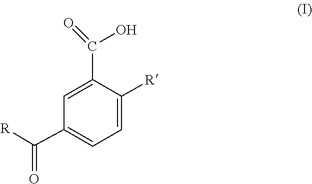

in which:
   the radical R denotes a linear, branched or cyclic, saturated aliphatic chain containing from 2 to 22 carbon atoms; an unsaturated chain containing from 2 to 22 carbon atoms containing one or more double bonds that may be conjugated; an aromatic nucleus linked to the carbonyl radical directly or via saturated or unsaturated aliphatic chains containing from 2 to 7 carbon atoms; said groups possibly being substituted with one or more substituents, which may be identical or different, chosen from halogen atoms, a trifluoromethyl group, hydroxyl groups in free form or esterified with an acid containing from 1 to 6 carbon atoms, or carboxyl groups in free form or esterified with a lower alcohol containing from 1 to 6 carbon atoms;
   R′ is a hydroxyl group or an ester group of formula:

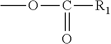

in which R₁ denotes a linear or branched, saturated or unsaturated aliphatic chain containing 1 to 18 carbon atoms;
and also salts thereof derived from an inorganic or organic base.

Preferably, the R radical in formula (I) denotes a linear, branched or cyclic, saturated aliphatic chain containing from 3 to 11 carbon atoms; an unsaturated chain containing from 3 to 17 carbon atoms and comprising one or more conjugated or unconjugated double bonds; it being possible for said hydrocarbon-based chains to be substituted with one or more substituents, which may be identical or different, chosen from halogen atoms, a trifluoromethyl group, hydroxyl groups in free form or esterified with an acid containing from 1 to 6 carbon atoms, or a carboxyl function in free form or esterified with a lower alcohol containing from 1 to 6 carbon atoms.

In one preferred embodiment, the R radical in formula (I) denotes a linear or branched alkyl or alkenyl group, preferably alkyl group, containing 2 or more, preferably 3 or more, more preferably 4 or more, even more preferably 5 or more, and in particular 6 or more carbon atoms, and/or 22 or less, preferably 18 or less, even more preferably 14 or less, preferentially 12 or less, and in particular 10 or less carbon atoms.

Preferentially, R′ in formula (I) is a hydroxyl group or an ester group of formula:

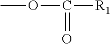

in which R₁ denotes a radical —(CH₂)ₙ—CH₃ where n is a number ranging from 0 to 14.

The salicylic acid derivatives that are more particularly preferred are those according to formula (I) in which the R radical is a C₃-C₁₀ alkyl group and/or R′ denotes hydroxyl.

Other particularly advantageous compounds are those in which R represents a chain derived from caprylic, linoleic, linolenic or oleic acid.

Another group of particularly preferred salicylic acid derivatives is constituted of compounds in which the R radical denotes a C₃-C₁₀ alkyl group bearing a carboxyl function in free form or esterified with a lower alcohol containing from 1 to 6 carbon atoms and R′ denotes hydroxyl.

The salicylic acid or its derivatives of formula (I) that may be used according to the present invention are in particular described in U.S. Pat. Nos. 6,159,479 and 5,558,871, FR 2,581,542, U.S. Pat. No. 4,767,750, EP 378 936, U.S. Pat. Nos. 5,267,407, 5,667,789, 5,580,549 and EP-A 570,230.

Among the particularly preferred salicylic acid derivatives of formula (I), mention may be made of 5-n-octanoylsalicylic acid (or capryloyl salicylic acid); 5-n-decanoylsalicylic acid; 5-n-dodecanoylsalicylic acid; 5-n-heptyloxysalicylic acid, and the corresponding salts thereof. The derivative in question is preferably 5-n-octanoylsalicylic acid.

For the purposes of the present invention, the salts of the salicylic acid or its derivatives are also considered. As the salts derived from inorganic bases, mention may particularly be made of those derived from alkali metal or alkaline-earth metal hydroxylated bases, for instance sodium hydroxide or potassium hydroxide, and ammonia. As regards the salts derived from the organic bases, mention may particularly be made of those derived from bases of amine or alkanolamine type.

The (b) salicylic acid or its derivative(s) may be present in an amount of from 0.1% to 5% by weight, preferably from 1% to 3%% by weight, relative to the total weight of the composition.

### (Cationic Polymer)

The cationic polymer may be a polysaccharide derived from β-linked D-glucosamine and N-acetyl-D-glucosamine. A particularly preferred cationic polymer is chitosan.

### (Thickener)

In some instances, the hair cosmetic compositions of the present disclosure include at least one stabilizer and/or thickening agent. The stabilizer and/or thickening agent(s) may be thickening polymers. Non-limiting examples include acacia, agar, algin, alginic acid, ammonium alginate, amylopectin, calcium alginate, sodium alginate, calcium carrageenan, carnitine, carrageenan, dextrin, gelatin, gellan gum, guar gum, tragacanth gum, acacia gum, Arabic gum, guar hydroxypropyltrimonium chloride, hectorite, hyaluronic acid, hydrated silica, hydroxypropyl chitosan, hydroxypropyl guar, karaya gum, kelp, locust bean gum, natto gum, potassium alginate, potassium carrageenan, propylene glycol alginate, sodium carboxymethyl dextran, sodium carrageenan, tragacanth gum, xanthan gum, modified xanthan gum, biosacharide gum, chitin, levan, elsinan, collagen, gelatin, zein, gluten, soy protein, casein, and mixtures thereof. In some cases, carrageenan is a particularly preferred stabilizer/thickening agent.

According to one preferred embodiment of the present invention, the weight ratio of the (a) glycolipid(s) to the (b) salicylic acid or its derivative(s) in the composition can be from 3:1 to 1:3, preferably from 2:1 to 1:2, more preferably 3:2.

In one preferred embodiment of the present invention, the composition comprises:
(a) at least one biosurfactant selected from rhamnolipids and sophorolipids; and
(b) at least one salicylic acid or its derivatives according to formula (I):

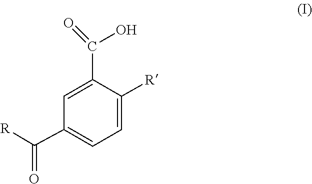

in which
the R radical denotes a linear or branched alkyl or alkenyl group, preferably alkyl group, containing 2 or more, preferably 3 or more, more preferably 4 or more, even more preferably 5 or more, and in particular 6 or more carbon atoms, and/or 22 or less, preferably 18 or less, even more preferably 14 or less, preferentially 12 or less, and in particular 10 or less carbon atoms, and R′ is a hydroxyl group;
(c) at least one cationic polymer may be a polysaccharide derived from β-linked D-glucosamine and N-acetyl-D-glucosamine; and
(d) at least one thickener.

### (Cosmetically Acceptable Hydrophilic Organic Solvent)

The composition according to the present invention may comprise at least one cosmetically acceptable hydrophilic organic solvent. The term “hydrophilic” here means substances having a solubility of at least 1 g/L, preferably at least 10 g/L, and more preferably at least 100 g/L, in water at room temperature (25° C.) and atmospheric pressure (10⁵ Pa).

The cosmetically acceptable hydrophilic organic solvent(s) may include, for example, substantially linear or branched lower mono-alcohols having from 1 to 8 carbon atoms, such as ethanol, propanol, butanol, isopropanol, and isobutanol; aromatic alcohols, such as benzyl alcohol and phenylethyl alcohol; polyols or polyol ethers, such as propylene glycol, dipropylene glycol, isoprene glycol, butylene glycol, glycerine, propanediol, caprylyl glycol, sorbitol, ethylene glycol monomethyl, monoethyl and monobutyl ethers, propylene glycol ethers, such as propylene glycol monomethylether, diethylene glycol alkyl ethers, such as diethylene glycol monoethylether or monobutylether; polyethylene glycols, such as PEG-4, PEG-6, and PEG-8, and their derivatives, and a combination thereof.

The amount of the cosmetically acceptable hydrophilic organic solvent(s) in the composition according to the present invention may be from 1% by weight or more, preferably 2% by weight or more, and more preferably 5% by weight or more, and may be 25% by weight or less, preferably 20% by weight or less, and more preferably 15% by weight or less, relative to the total weight of the composition.

### (pH Adjusting Agent)

The pH of the composition according to the present invention may be adjusted to the desired value using at least one pH adjusting agent, such as an acidifying or a basifying agent, for example, which are commonly used in cosmetic products.

The pH of the composition according to the present invention may be from 9 to 3, more preferably from 7 to 4, and even most preferably from 6 to 5. Particularly, the most preferred pH is 5.5.

Among the acidifying agents, mention may be made, by way of example, of mineral or organic acids such as hydrochloric acid, ortho-phosphoric acid, sulfuric acid, carboxylic acids such as acetic acid, tartaric acid, citric acid, and lactic acid, and sulfonic acids.

Among the basifying agents, mention may be made, by way of example, of hydroxides of an alkali metal or an alkaline-earth metal, for instance sodium hydroxide or potassium hydroxide; quaternary ammonium hydroxides and guanidinium hydroxide; alkali metal silicates, such as sodium metasilicates; amino acids, preferably basic amino acids, such as arginine, lysine, ornithine, citrulline and histidine; carbonates and bicarbonates, particularly of a primary amine, secondary amine or tertiary amine, of an alkali metal or alkaline-earth metal, or of ammonium; and the compounds of the following formula:

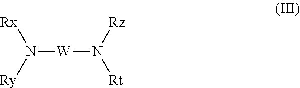

in which
W is a C₁-C₆ alkylene residue optionally substituted with a hydroxyl group or a C₁-C₆ alkyl group; Rx, Ry, Rz and Rt, which may be identical or different, represent a hydrogen atom or a C₁-C₆ alkyl, C₁-C₆ hydroxyalkyl or C₁-C₆ aminoalkyl group. Mention may especially be made of 1,3-diaminopropane, 1,3-diamino-2-propanol, spermine and spermidine.

The pH adjusting agent(s) may be used in an amount ranging from 0.001% to 10% by weight, preferably from 0.01% to 5% by weight, and more preferably from 0.1% to 3% by weight, relative to the total weight of the composition.

### (Water)

The composition according to the present invention preferably includes water.

The amount of water in the composition according to the present invention may be from 20% to 70%% by weight, relative to the total weight of the composition.

### (Optional Additives)

The composition according to the present invention may also comprise any optional additive(s) usually used in the field of cosmetics, chosen, for example, from anionic, cationic, nonionic or amphoteric polymers, anionic, cationic, nonionic or amphoteric surfactants, oils, hydrophobic organic solvents, gums, resins, thickeners, dispersants, antioxidants, buffers, organic or inorganic fillers, preserving agents, such as phenoxyethanol, fragrances, neutralizers, antiseptics, UV-screening agents, cosmetic active agents such as vitamins, moisturizers, emollients or collagen-protecting agents, and mixtures thereof.

As the antiseptics, those generally used for cosmetics can be used. As the specific examples of the antiseptics, mention can be made of, for example, ethanol, triclosan, piroctone olamine, triclocarban, paraben, acrinol, alkyldiaminoglycine or a salt thereof, povidone iodine, potassium iodide, iodine, 1,2-pentanediol, halocarban, 3,4,4-trichlorocarbanilide, triethyl citrate, resorcin, hexachlorophene, silver-supported zeolite, silver-supported silica, gluconic acid, benzoic acid, sodium benzoate, undecylene acid, salicylic acid, sorbic acid or a salt thereof, dehydroacetic acid or a salt thereof, methyl paraoxybenzoate, ethyl paraoxybenzoate, propyl paraoxybenzoate, isopropyl paraoxybenzoate, butyl paraoxybenzoate, isobutyl paraoxybenzoate, benzyl paraoxybenzoate; phenols such as isopropylmethylphenol, chlorhexidine gluconate, cresol, timole, parachlorophenol, phenylethyl alcohol, phenylphenol, sodium phenylphenol, sodium phenoxyethanol, phenoxydiglycol, phenol, benzyl alcohol; tertiary ammonium salt compounds such as stearyltrimethylammonium chloride, cetyltrimethylammonium chloride, cetylpyridinium chloride, benzalconium chloride, benzethonium chloride, methylbenzethonium chloride, lauryltrimethylammonium chloride, alkylisoquinolium bromide, domiphenone bromide; plant extracts such as tea extract, hinokithiol, apple extract; chloramine T, chlorhexidine, benzoic acidion, and mixtures thereof.

The antiseptic(s) may be used in an amount ranging from 0.001% to 10% by weight, preferably from 0.01% to 5% by weight, and more preferably from 0.1% to 3% by weight, relative to the total weight of the composition.

The composition according to the present invention can be prepared by mixing the above-described essential and optional ingredients in a conventional manner. In the case that at least one of the above ingredients is solid at room temperature, the ingredient can be heated until it is dissolved. It is possible to further comprise mixing any of the optional ingredients and heating the composition until an ingredient is dissolved.

(Method for Treating, Hydrating and/or Cleansing Acne-Prone Skin)

The present invention also relates to a method for treating, hydrating, and/or cleansing as acne-prone skin including scalp, lips, in particular facial skin, comprising:
applying onto the keratinous substance a composition comprising:
   (a) at least one glycolipid;
   (b) at least one salicylic acid or its derivatives of formula (I) above;
   (c) at least one cationic polymer; and
   (d) at least one thickener.

Treating acne-prone skin in present invention means reduction of sebum, or deposition of salicylic acid or its derivatives, or reduction of viable cells of Cutibacterium acnes.

Cleanability of acne-prone skin is calculated as below:

$$Cleansability = \frac{{De}_{sample cleaned} - {De}_{black reference}}{{De}_{clean reference} - {De}_{black reference}}$$

$${D}_{e} = \sqrt{{D}_{l}^{2} + {D}_{a}^{2} + {D}_{b}^{2}}$$

De: color difference in a rectangular coordinate system
Black reference: bioskin with sebollution applied following pore deep clean protocol
Clean reference: clean bioskin w/o any sebollution

The composition is generally applied on a keratinous substance, such as skin, with the hands or an applicator. The present invention may comprise an optional step of rinsing the composition from the keratinous substance after it is applied.

The same explanations for the composition
(a) at least one glycolipid;
(b) at least one salicylic acid or its derivatives of formula (I) above;
(c) at least one cationic polymer; and
(d) at least one thickener
   as the composition according to the present invention above can be applied to the composition,
(a) at least one glycolipid;
(b) at least one salicylic acid or its derivatives of formula (I) above;
(c) at least one cationic polymer; and
(d) at least one thickener
   for the method

The composition used in the method according to the present invention may include any of the optional ingredients as explained above for the composition according to the present invention.

### EXAMPLES

The present invention will be described in more detail by way of examples which however should not be construed as limiting the scope of the present invention.

### Examples 1 to 11 of Present Invention

Compositions of Examples 1 to 11 were prepared by mixing the ingredients as listed in Table 1 with a magnetic stirrer. The numerical values for the amounts of the ingredients shown in Table 1 are all based on “% by weight” as active raw materials. Rhamnolipids were obtained from EVONIK (trade name: RHEANCE One).

### [Hydration Study Results]

Examples 8-11 were evaluated against petroleum jelly (positive control) and bare skin for hydration. Subjects cleansed each volar forearm with one 70% isopropyl alcohol wipe. Skin was permitted to air dry for thirty (30) minutes prior to the baseline measurements, with the volar forearms exposed in a room with controlled temperature and humidity conditions. Baseline measurements of each site were taken with the Corneometer 825. Additional measurements were taken at 15 minutes and one hour.

TABLE 1

| Ingredient | Function | Ex. 1 | Ex. 2 | Ex. 3 | Ex. 4 | Ex. 5 | Ex. 6 | Ex. 7 | Ex. 8 | Ex. 9 | Ex. 10 | Ex. 11 |
|---|---|---|---|---|---|---|---|---|---|---|---|---|
| Sodium Hydroxide | Active | 0.350 | 0.350 | 0.300 | 0.300 | 0.300 | 0.300 | 0.350 | 0.450 | 0.450 | 0.250 | 0.450 |
| | Compound | | | | | | | | | | | |
| Tartaric Acid | Active | | | | 0.200 | | | | | | | |
| | Compound | | | | | | | | | | | |
| Lactic Acid | Active | 0.050 | 0.050 | | | 0.050 | 0.050 | 0.050 | 0.050 | 0.050 | 0.050 | 0.050 |
| | Compound | | | | | | | | | | | |
| Trisodium | Active | 0.500 | 0.500 | 0.500 | | 0.500 | 0.500 | 0.500 | 0.500 | 0.500 | 0.500 | 0.500 |
| Ethylenediamine | Compound | | | | | | | | | | | |
| Disuccinate | | | | | | | | | | | | |
| Sodium Lauroyl | Active | 0.250 | 0.250 | 0.250 | 0.250 | 0.250 | 0.250 | 0.250 | 0.250 | 0.250 | 0.250 | 0.250 |
| Lactylate (and) | Compound | | | | | | | | | | | |
| Mixture of | | | | | | | | | | | | |
| Ceramides (and) | | | | | | | | | | | | |
| Phytosphingosine | | | | | | | | | | | | |
| (and) Cholesterol | | | | | | | | | | | | |
| (and) Xanthan Gum | | | | | | | | | | | | |
| (and) Carbomer | | | | | | | | | | | | |
| Cetyl Alcohol | Fatty | 2.500 | 2.500 | 2.500 | 2.500 | 2.500 | 2.500 | 2.500 | 2.500 | 2.500 | 2.500 | 2.500 |
| | Compound | | | | | | | | | | | |
| Kaolin | Filler | 15.0 | 15.0 | 15.0 | 15.0 | 15.0 | 15.0 | 15.0 | 10.0 | 15.0 | 15.0 | 15.0 |
| Carrageenan | Polymer | 1.0 | 1.0 | 1.0 | 1.0 | 1.0 | 1.0 | 1.0 | 0.8 | 1.0 | 1.0 | 1.0 |
| Chitosan | Polymer | 0.10 | 0.10 | 0.10 | 0.10 | 0.10 | 0.10 | 0.10 | 0.10 | 0.10 | 0.10 | 0.10 |
| Salicylic Acid | Active | 2.0 | 2.0 | 2.0 | 2.0 | 2.0 | 2.0 | 2.0 | 2.0 | 2.0 | 2.0 | 2.0 |
| Phenoxyethanol | Preservative | 0.30 | | | | | | 0.30 | | | | |
| Water | Solvent | 55.95 | 51.25 | 56.35 | 56.65 | 51.30 | 56.30 | 55.95 | 23.35 | 48.15 | 48.35 | 28.15 |
| Glycerin | Solvent | 2.0 | 2.0 | 2.0 | 2.0 | 2.0 | 2.0 | 2.0 | 40.0 | 10.0 | 10.0 | 30.0 |
| Glyceryl Stearate SE | Surfactant | 5.0 | 5.0 | 5.0 | 5.0 | 5.0 | 5.0 | 5.0 | 5.0 | 5.0 | 5.0 | 5.0 |
| Sodium Cocoyl | Surfactant | | 10.0 | | | 10.0 | 10.0 | | 10.0 | 10.0 | 10.0 | 10.0 |
| Isethionate | | | | | | | | | | | | |
| Sodium Methyl | Surfactant | 10.0 | 5.0 | 10.0 | 10.0 | 5.0 | | 10.0 | | | | |
| Cocoyl Taurate | | | | | | | | | | | | |
| Rhamnolipids | Surfactant | 3.0 | 3.0 | 3.0 | 3.0 | 3.0 | 3.0 | 3.0 | 3.0 | 3.0 | 3.0 | 3.0 |
| Niacinamide | Active | 2.0 | 2.0 | 2.0 | 2.0 | 2.0 | 2.0 | 2.0 | 2.0 | 2.0 | 2.0 | 2.0 |

The results of the hydration study are provided in Table 2, with the values indicating the level of hydration at baseline, 15 minutes, and 1 hour. As shown in Table 2, Examples 8 through 11 were effective for hydrating the skin at 15 minutes and one-hour intervals, compared to the baseline as well as evaluated against petroleum jelly (positive control) and bare skin.

TABLE 2

| Example | Baseline | 15 min | 1 hr |
|---|---|---|---|
| Petroleum Jelly | 31.47 ± 4.60 | 37.00 ± 9.34 | 39.36 ± 8.06 |
| Bare (Wet) | 31.92 ± 4.82 | 30.37 ± 7.10 | 30.88 ± 7.09 |
| Ex. 8 | 31.54 ± 5.40 | 45.47 ± 9.30 | 45.71 ± 10.23 |
| Ex. 10 | 30.12 ± 5.36 | 32.28 ± 7.64 | 32.83 ± 7.70 |
| Ex. 9 | 29.16 ± 4.94 | 36.07 ± 8.14 | 35.38 ± 8.35 |
| Ex. 11 | 30.28 ± 4.98 | 37.58 ± 7.42 | 38.37 ± 8.78 |

### Examples 12 to 16 of Present Invention

Compositions of Examples 12 to 19 were prepared by mixing the ingredients as listed in Table 3 with a magnetic stirrer. The numerical values for the amounts of the ingredients shown in Table 3 are all based on “% by weight” as active raw materials. Rhamnolipids were obtained from EVONIK (trade name: RHEANCE One).

TABLE 3

| INCI name | Cosmetic type | Ex 12 | Ex 13 | Ex 14 | Ex 15 | Ex 16 | Ex 17 | Ex 18 | Ex 19 |
|---|---|---|---|---|---|---|---|---|---|
| sodium hydroxide | active compound | 0.45 | 0.40 | 0.45 | 0.35 | 0.30 | 0.30 | 0.45 | 0.45 |
| niacinamide | active compound | 2.00 | 2.00 | 2.00 | 2.00 | 2.00 | 2.00 | 2.00 | 2.00 |
| glyceryl stearatese | surfactant | 5.00 | 5.00 | 5.00 | 5.00 | 5.00 | 5.00 | 5.00 | 5.00 |
| carrageenan | polymer | 1.00 | 1.00 | 1.00 | 1.00 | 1.00 | 1.00 | 0.80 | 1.00 |
| salicylic acid | active compound | 2.00 | 2.00 | 2.00 | 2.00 | 2.00 | 2.00 | 2.00 | 2.00 |
| phenoxyethanol | preservative | | | | 0.30 | | | | |
| water | solvent | 28.15 | 47.70 | 48.15 | 55.95 | 51.30 | 56.30 | 23.35 | 38.15 |
| glycerin | solvent | 30.00 | 10.00 | 10.00 | 2.00 | 2.00 | 2.00 | 40.00 | 20.00 |
| lactic acid | active compound | 0.05 | 0.05 | 0.05 | 0.05 | 0.05 | 0.05 | 0.05 | 0.05 |
| cetyl alcohol | fatty compound | 2.50 | 2.50 | 2.50 | 2.50 | 2.50 | 2.50 | 2.50 | 2.50 |
| kaolin | filler | 15.00 | 15.00 | 15.00 | 15.00 | 15.00 | 15.00 | 10.00 | 15.00 |
| sodium cocoyl | surfactant | 10.00 | 10.00 | 10.00 | | 10.00 | 10.00 | 10.00 | 10.00 |
| isethionate | | | | | | | | | |
| trisodium | active compound | 0.50 | 1.00 | 0.50 | 0.50 | 0.50 | 0.50 | 0.50 | 0.50 |
| ethylenediamine | | | | | | | | | |
| disuccinate | | | | | | | | | |
| sodium lauroyl | active compound | 0.25 | 0.25 | 0.25 | 0.25 | 0.25 | 0.25 | 0.25 | 0.25 |
| lactylate (and) | | | | | | | | | |
| ceramide np (and) | | | | | | | | | |
| ceramide ap (and) | | | | | | | | | |
| phytosphingosine | | | | | | | | | |
| (and) cholesterol | | | | | | | | | |
| (and) xanthan | | | | | | | | | |
| gum (and) | | | | | | | | | |
| carbomer (and) | | | | | | | | | |
| ceramide eop | | | | | | | | | |
| glycolipids | surfactant | 3.00 | 3.00 | 3.00 | 3.00 | 3.00 | 3.00 | 3.00 | 3.00 |
| chitosan | polymer | 0.10 | 0.10 | 0.10 | 0.10 | 0.10 | 0.10 | 0.10 | 0.10 |
| sodium methyl | surfactant | | | | 10.00 | 5.00 | | | |
| cocoyl taurate | | | | | | | | | |

### [Sebum Control Study Results]

Sebum control studies were conducted according to the following procedure:
1. Wash face with water & acclimatization in standard condition for 30 mins
2. T0 baseline measurement on Lightcam
3. Product application as per randomization using standard application gesture
4. Subjects to wait in standard condition for 10 mins
5. T imm measurement on Lightcam
6. Subjects to wait in standard conditions for next 4 hrs for subsequent instrumental measurements

The sebum control study results shown in Table 4 indicates that Examples 12 and 13 had significant reduction of sebum, which outperformed some of the best benchmark products on the markets.

TABLE 4

| | Change in gloss shine |
|---|---|
| Ex 13 | −2.47 |
| Ex 12 | −2.48 |

### [Cleansability Study Results]

Cleansability studies were carried out according to the procedure illustrated below. Briefly, first apply the sebollution and take a picture for T0, then dilute the cleanser and apply it on sebollution, and finally massage, take another picture at T1.

The calculation for cleansability is also illustrated below. The results are shown in Table 5.

As shown in Table 5, Examples 17, 18, 14, and 12 achieved good cleansability, comparable to the cleansability of benchmark cleansing products on the markets.

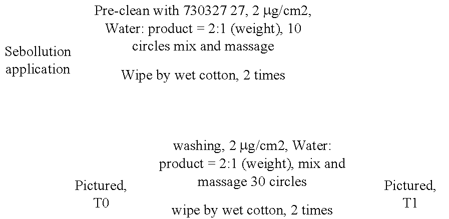

$$Cleansability = \frac{{De}_{sample cleaned} - {De}_{black reference}}{{De}_{clean reference} - {De}_{black reference}}$$

$${D}_{e} = \sqrt{{D}_{l}^{2} + {D}_{a}^{2} + {D}_{b}^{2}}$$

De: color difference in a rectangular coordinate system
Black reference: bioskin with sebollution applied following pore deep clean protocol
Clean reference: clean bioskin w/o any sebollution

TABLE 5

| | % |
|---|---|
| | Cleansability |
| Ex 17 | 67 |
| Ex 18 | 72 |
| Ex 14 | 88 |
| Ex 12 | 85 |

### [C. Acne Study Results]

The C. acne studies were performed according to the Microbiology department standard procedure I_M_ANA_006_01 “Activity of formulae against microorganisms of cosmetic interest”.

In summary, in a suitable culture medium, we evaluated the biocidal effect on the strain of interest of formulae at 10% after 2 h, 6 h and 24 hours of contact time, in comparison to a control (medium without formula). The reduction in the number of viable bacteria is measured over time. This assay is a “Time Kill” test.

The C. acne results for log reduction compared to the control are shown in Table 6. The C. acne study results for Examples 17, 18, 14, and 12 indicate satisfactory level of reduction.

Note that Examples 17, 18, 14, and 12 had a pH around 5.5. We further observed that the pH of the samples had significant effect on C. acne reduction. The C. acne results for log reduction with effect of pH are shown in Table 7. A lower pH of 5.5 clearly improved the C. acne reduction compared to pH 7.2.

TABLE 6

| | | 2 H | 6 H | 24 H |
|---|---|---|---|---|
| Ex 17 | Acne Paste Cleanser 2% Glycerin | 0.6 | 1 | 2.3 |
| Ex 18 | Acne Paste Cleanser 40% Glycerin | 0.4 | 0.8 | 2 |
| Ex 14 | Acne Paste Cleanser 10% Glycerin | 0.6 | 0.9 | 2 |
| Ex 12 | Acne Paste Cleanser 30% Glycerin | 0.6 | 1 | 2 |

TABLE 7

| | | | Log reduction compared | | |
|---|---|---|---|---|---|
| | | | to the Control | | |
| | | | 2 H | 6 H | 24 H |
| PH | Ex 16 | Acne Paste Cleanser 2% | 1.9 | 3.9 | 5 |
| 5.5 | | Glycerin + 10% Sodium Cocoyl | | | |
| | | Isethionate | | | |
| | Ex 15 | Acne Paste Cleanser 2% Glycerin | 1.5 | 1.4 | 4.1 |
| pH | Ex 16 | Acne Paste Cleanser 2% | 0 | 0.2 | 0.6 |
| 7.2 | | Glycerin + 10% Sodium Cocoyl | | | |
| | | Isethionate | | | |
| | Ex 15 | Acne Paste Cleanser 2% Glycerin | 0 | 0 | 0 |

1. A cosmetic composition comprising:
(a) at least one glycolipid;
(b) at least one salicylic acid or its derivatives of formula (I): 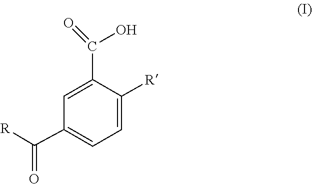
in which:
the radical R denotes a linear, branched or cyclic, saturated aliphatic chain containing from 2 to 22 carbon atoms; an unsaturated chain containing from 2 to 22 carbon atoms containing one or more double bonds that may be conjugated; an aromatic nucleus linked to the carbonyl radical directly or via saturated or unsaturated aliphatic chains containing from 2 to 7 carbon atoms; said groups possibly being substituted with one or more substituents, which may be identical or different, chosen from halogen atoms, a trifluoromethyl group, hydroxyl groups in free form or esterified with an acid containing from 1 to 6 carbon atoms, or carboxyl groups in free form or esterified with a lower alcohol containing from 1 to 6 carbon atoms;
R′ is a hydroxyl group or an ester group of formula: 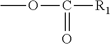
in which R₁ denotes a linear or branched, saturated or unsaturated aliphatic chain containing 1 to 18 carbon atoms;
and also salts thereof derived from an inorganic or organic base;
(c) at least one cationic polymer selected from a polysaccharide derived from β-linked D-glucosamine or N-acetyl-D-glucosamine; and
(d) at least one thickener.

2. The composition according to claim 1, wherein the glycolipid is selected from the group consisting of rhamnolipids, sophorolipids, glucolipids, trehalolipids, cellobiose lipids, and mixtures thereof.

3. The composition according to claim 2, wherein the glycolipid is rhamnolipids.

4. The composition according to claim 1, wherein the salicylic acid derivatives are represented by formula (I) in which the radical R denotes a linear, branched or cyclic, saturated aliphatic chain containing from 2 to 22 carbon atoms.

5. The composition according to claim 1, wherein the salicylic acid derivatives are represented by formula (I) in which the R radical denotes a linear or branched alkyl or alkenyl group, preferably alkyl group, containing 2 or more, preferably 3 or more, more preferably 4 or more, even more preferably 5 or more, and in particular 6 or more carbon atoms, and/or 22 or less, preferably 18 or less, even more preferably 14 or less, preferentially 12 or less, and in particular 10 or less carbon atoms.

6. The composition according to claim 1, wherein the salicylic acid derivatives are represented by formula (I) in which R′ denotes a hydroxyl group.

7. The composition according to claim 1, wherein the salicylic acid or its derivatives is salicylic acid.

8. The composition according to claim 1, wherein the amount of the glycolipid(s) is from 0.1% by weight to 10% by weight, relative to the total weight of the composition.

9. The composition according to claim 8, wherein the amount of the glycolipid(s) is from 1% by weight to 7% by weight, relative to the total weight of the composition.

10. The composition according to claim 9, wherein the amount of the glycolipid(s) is from 2% by weight to 5% by weight, relative to the total weight of the composition.

11. The composition according to claim 1, wherein the amount of the salicylic acid or its derivatives is from 0.1% by weight to 5% by weight, relative to the total weight of the composition.

12. The composition according to claim 5, wherein the amount of the salicylic acid or its derivatives is from 1% by weight to 3% by weight, relative to the total weight of the composition.

13. The composition according to claim 1, wherein the weight ratio of the glycolipid(s) to the salicylic acid or its derivatives in the composition is from 3:1 to 1:3.

14. The composition according to claim 1, wherein the weight ratio of the glycolipid(s) to the salicylic acid or its derivatives in the composition is from 2:1 to 1:2.

15. The composition according to claim 1, wherein the thickener is a plant-based thickener.

16. The composition according to claim 1, wherein the cationic polymer is chitosan.

17. The composition according to claim 1, wherein the composition is in the form of a paste.

18. The composition according to claim 1, wherein the composition further includes water in an amount of 20% by weight to 70% by weight, relative to the total weight of the composition.

19. The composition according to claim 1, wherein the composition is a cosmetic composition for treating, hydrating, and/or cleansing acne-prone skin.

20. A method for treating, hydrating, and/or cleansing acne-prone skin, comprising:
applying onto the skin in need a composition comprising:
(a) at least one glycolipid; and
(b) at least one salicylic acid or its derivatives of formula (I): 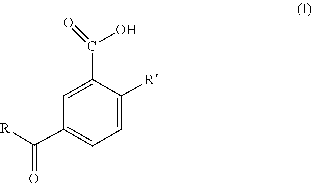
in which:
the radical R denotes a linear, branched or cyclic, saturated aliphatic chain containing from 2 to 22 carbon atoms; an unsaturated chain containing from 2 to 22 carbon atoms containing one or more double bonds that may be conjugated; an aromatic nucleus linked to the carbonyl radical directly or via saturated or unsaturated aliphatic chains containing from 2 to 7 carbon atoms; said groups possibly being substituted with one or more substituents, which may be identical or different, chosen from halogen atoms, a trifluoromethyl group, hydroxyl groups in free form or esterified with an acid containing from 1 to 6 carbon atoms, or carboxyl groups in free form or esterified with a lower alcohol containing from 1 to 6 carbon atoms;
R′ is a hydroxyl group or an ester group of formula: 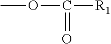
in which R₁ denotes a linear or branched, saturated or unsaturated aliphatic chain containing 1 to 18 carbon atoms;
and also salts thereof derived from an inorganic or organic base;
(c) at least one cationic polymer may be a polysaccharide derived from β-linked D-glucosamine and N-acetyl-D-glucosamine; and
(d) at least one plant-based thickener.
